(19) United States
(12) Patent Application Publication
(10) Pub. No.: US 2011/0046420 A1
(43) Pub. Date: Feb. 24, 2011

(54) PROCESS FOR THE CO-PRODUCTION OF ALCOHOLS

(75) Inventors: Kurt W. KRAMARZ, Murrysville, PA (US); Richard J. Ingram, McDonald, PA (US); John E. Aiken, Monroeville, PA (US); George R. Gallaher, Oakmont, PA (US)

Correspondence Address: NIXON & VANDERHYE, PC, 901 NORTH GLEBE ROAD, 11TH FLOOR, ARLINGTON, VA, 22203, US

(73) Assignee: BASF Corporation

(21) Appl. No.: 12/870,447

(22) Filed: Aug. 27, 2010

Related U.S. Application Data
(63) Continuation of application No. 10/716,920, filed on Nov. 19, 2003, now abandoned.
(60) Provisional application No. 60/439,730, filed on Jan. 13, 2003.

(51) Int. Cl. C07C 29/14

(52) U.S. Cl. 568/881

(57) ABSTRACT

The present invention relates to the co-production of unsaturated aldehydes via a crossed-aldol condensation reaction catalyzed by recyclable water-soluble phase-transfer catalysts or the hydroxides thereof. The aldehydes are then hydrogenated to the desired alcohol products or saturated aldehyde feed stocks. Specifically, methods in which 2,4-diethyloctanol is co-produced with 2-ethylhexanol in batch and continuous processes are described. Recovery of the phase-transfer catalyst through water washing followed by “salting out” from the washings is also demonstrated.

### REFERENCE TO RELATED APPLICATIONS

This is a continuation of commonly owned copending U.S. application Ser. No. 10/716, 920, filed Nov. 19, 2003, which is based on and claims domestic priority benefits under 35 USC §119(e) from U.S. Provisional Application Ser. No. 60/439,730 filed on Jan. 13, 2003, the entire content of each being expressly incorporated hereinto by reference.

### FIELD OF THE INVENTION

The present invention relates to the co-production of unsaturated aldehydes via a crossed-aldol reaction catalyzed by recyclable water-soluble phase-transfer catalysts or the hydroxides thereof. The aldehydes are hydrogenated to the desired alcohol products or saturated aldehyde feed stocks. Specifically, a process in which 2,4-diethyloctanol is co-produced with 2-ethylhexanol is described.

### BACKGROUND OF THE INVENTION

It is well known that the process for 2-ethylhexanol production makes use of propylene hydroformylation to make n-butyraldehyde, which undergoes a sclf-aldol condensation with the elimination of water to produce the intermediate 2-ethyl-2-hexenal. Both the olefin and aldehyde functionalities of 2-ethyl-2-hexenal are then hydrogenated to yield the saturated alcohol, 2-ethylhexanol. World-wide annual capacity of 2-ethylhexanol has been estimated at over 3 million tons (Cornils et al., “Applied Homogeneous Catalysis with Organometallic Compounds”, Wiley-VCH 2002). The plasticizer industry is the major consumer of 2-ethylhexanol with other significant consumption accounted for by 2-ethylhexyl acrylate, diesel fuel additives, and lube oil additive production.

Plasticizer alcohols of even higher molecular weight than 2-ethylhexanol are desired for the manufacture of plasticizers with lower volatilities. These less volatile plasticizers are better suited for applications that require greater permanence in higher demand applications. Such plasticizer alcohols can be limited by their compatibility with the polymer into which they are compounded (e.g. polyvinyl chloride or PVC). Plasticizer alcohols of greater molecular weight than 2-ethylhexanol which are suitable for PVC plasticizers are isononyl, isodecyl, undecyl, and tridecyl alcohols. These alcohols are produced from the hydroformylation of the following higher olefins respectively: octenes, nonenes, decenes, and dodecenes. In general, these olefins can be more costly than propylene and the alcohols are normally more expensive than 2-ethylhexanol. The hydroformylation of these higher olefin feeds requires higher temperatures and pressures than employed by the typical propylene hydroformylation in a 2-ethylhexanol plant, which makes use of low pressure phosphine-modified rhodium- or cobalt-catalyzed systems.

2,4-diethyloctanol is a higher alcohol, which has been previously observed in small amounts in 2-ethylhexanol and di(2-ethylhexyl)phthalate production (Upadysheva et al., USSR Deposited Doc. 1982). The use of tricaprylmethylammonium chloride as a phase-transfer catalyst in an aldol reaction has been previously described (GB 1547856, GB 1547857) to yield 2,4-diethyl-2-octenal and its subsequent hydrogenation to 2,4-diethyloctanol. While soluble in the aldehyde phase, tricaprylmethylammonium chloride is not readily water soluble and forms an emulsion upon mixing with water or an aqueous sodium hydroxide solution.

Prior art has shown that bis(2,4-diethyloctyl)phthalate prepared from 2,4-diethyloctanol has demonstrated valued plasticizer properties (SU347336, SU385323, Rabinovich, et al. Acta Polymerica, 1983, 482). A process which co-produces 2,4-diethyloctanol with 2-ethylhexanol is desirable as a source of low-cost plasticizer alcohols.

### SUMMARY OF INVENTION

The present invention relates to the co-production of unsaturated aldehydes via a crossed-aldol condensation reaction catalyzed by recyclable water-soluble phase-transfer catalysts or the hydroxides thereof. The improved process produces an unsaturated aldehyde reaction product by an aqueous base-catalyzed crossed-aldol condensation reaction between a first aldehyde containing 3-5 carbons and a second aldehyde containing 6-11 carbons. Examples of the first aldehyde can be, but are not limited to, propionaldehyde, n-butyraldehyde, isovaleraldehyde, or valeraldehyde. The aldehydes produced in the aldol condensation reactions are then hydrogenated to the desired alcohol products or saturated aldehyde feed stocks. Specifically, a process in which 2,4-diethyloctanol is co-produced with 2-ethylhexanol from n-butyraldehyde and 2-ethylhexanal is described.

There are two major improvements resulting from the use of a water-soluble phase-transfer catalyst. The phase-transfer catalyst improves the solubility of the hydroxide catalyst necessary for the crossed-aldol reaction in the longer chain aldehyde, enhancing selectivity for the desired crossed-aldol product versus the self-aldol product produced from the reaction of the shorter chain, more reactive aldehyde. Long chain aldehydes of six carbons or greater such as 2-ethylhexanal are reacted with shorter chain aldehydes such as n-butyraldehyde to form 2,4-diethyl-2-octenal in a crossed-aldol condensation with the major co-product from the self reaction of the n-butyraldehyde being 2-ethyl-2-hexenal. The excellent water solubility of phase-transfer catalysts such as tetrabutylammonium and methyltributylammonium chloride, bromide, or hydroxide derivatives allow for the second major improvement which is the facile recovery of the phase-transfer catalysts from the organic product by aqueous washing.

An added benefit of using the quaternary ammonium phase-transfer catalysts described is their phase separation from solutions of high alkalinity. The “salting out” of the phase-transfer catalysts allows their recovery from aqueous solutions for recycle and also facilitates their transfer into the organic phase from aqueous phase in the aldol reaction.

The higher aldehydes produced in the described process, such as 2,4-diethyl-2-octenal and the co-product 2-ethyl-2-hexenal, can be hydrogenated to alcohols suitable for use in the manufacture of plasticizers. Specifically, 2,4-diethyloctanol and 2-ethylhexanol can be used to manufacture adipate, maleate, phthalate, and trimellitate ester plasticizers. Examples of the application of this process to existing 2-ethylhexanol plants are described.

According to especially preferred embodiments of the present invention, an improved process is provided for the preparation of at least one primary alcohol by the hydrogenation of an unsaturated aldehyde reaction product produced by an aqueous base-catalyzed crossed-aldol reaction between a first aldehyde containing 3-5 carbons and a second aldehyde containing 6-11 carbons, wherein the selectivity of the crossed-aldol condensation reaction is enhanced through the use of a water-soluble phase-transfer catalyst. Preferably, the 3-5 carbon aldehyde is propionaldehyde, n-butyraldehyde, isovaleraldehyde, or valeraldehyde.

In some embodiments, the 2,4-diethyloctanol is produced concurrently with 2-ethylhexanol via the hydrogenation of 2-ethyl-2-hexenal and 2,4-diethyl-2-octenal produced form an aldol condensation reaction, which makes use of n-butyraldehyde and 2-ethylhexanal as the reactant aldehydes. Preferably, the molar ratio of 2-ethylhexanal to n-butyraldehyde fed to the crossed-aldol condensation reaction is about 1 to about 10, more preferably about 1 to about 5. In certain embodiments, the 2-ethylhexanal is produced by the partial hydrogenation of 2-ethyl-2-hexenal using a Group VIII metal catalyst, wherein the 2-ethyl-2-hexenal being produced by an aldol condensation reaction of n-butyraldehyde. A portion of the unreacted 2-ethylhexanal and 2-ethyl-2-hexenal may be covered from the crossed-aldol condensation reaction product in preference to hydrogenation to 2-ethylhexanol. In such embodiments, the 2-ethylhexanal is produced by the Group VIII metal catalyzed partial hydrogenation of the recovered 2-ethyl-2-hexenal. An especially preferred Group VII metal is palladium.

According to other preferred embodiments of the present invention, the water soluble phase transfer catalyst is quaternary ammonium or phosphonium salt.

One preferred embodiment of the present invention comprises removing the phase-transfer catalyst from the reaction product by water washing. Preferably the phase-transfer catalyst is recovered from the water washing by the addition of an alkali metal hydroxide to the water washing to a concentration of 2.5 to 12.5 molar, thereby producing a first phase containing the majority of the phase-transfer catalyst and a second aqueous alkali metal hydroxide phase. The alkali metal hydroxide is most preferably sodium hydroxide.

The cationic portion of the phase-transfer catalyst is preferably methyltributylammonium, tetrabutylammonium, benzyltriethylammonium, ethyltributylammonium, tetraethylammonium, tetrahexylammonium, tetrapropylammonium, or tetrabutylphosphonium. The anionic portion of the phase-transfer catalyst is preferably chloride, bromide, iodide, bisulfate, sulfate, or hydroxide. The aqueous base is preferably an alkali metal hydroxide, such as sodium hydroxide or potassium hydroxide. In some embodiments, the aqueous base comprises a 10-50 weight percent solution of sodium hydroxide. In other embodiments, the aqueous base is the hydroxide form of a quaternary ammonium or phosphonium salt.

The aldol reaction is preferably performed at a temperature from about 30 to 120° C., more preferably from about 30 to about 100° C. In some preferred embodiments, the crossed-aldol reaction takes place in a two-phase system comprising a first organic aldehyde phase and a second aqueous phase, and the phase-transfer catalyst is primarily in the organic aldehyde phase. In other preferred embodiments, the crossed-aldol reaction takes place in a three-phase system comprising a first organic aldehyde phase, a second aqueous phase, and a third phase containing the majority of the phase-transfer catalyst.

Preferably, the molar ratio of the phase-transfer catalyst to the first aldehyde is about 0.01 to about 2, more preferably about 0.1 to about 2.

The aldol reaction may performed in a continuous or batch reactor. The unsaturated aldehyde reaction product may be hydrogenated in the gas and/or liquid phase in a single or multistage process.

### BRIEF DESCRIPTION OF DRAWINGS

FIG. 1. A process flow diagram of the aldol and hydrogenation sections of a typical 2-ethylhexanol plant, 2=n-butyraldehyde; 4=aqueous sodium hydroxide; 6=2-Ethyl-2-hexenal; 8=2-Ethylhexanol.

FIG. 2. A process flow diagram of a 2-ethylhexanol plant modified with a selective hydrogenation unit and phase transfer catalyst recycle unit, 2=n-butyraldehyde; 4=aqueous sodium hydroxide; 6=2-Ethyl-2-hexenal; 8=2-Ethylhexanol, 10=2-ethylhexanal, 12=2,4-diethyl-2-octenal, 14=2,4-diethyloctanol, PTC=phase transfer catalyst.

FIG. 3. A process flow diagram of a 2-ethylhexanol plant modified with a selective hydrogenation unit and “quaternary hydroxide” recycle unit, 2=n-butyraldehyde; 4 aqueous sodium hydroxide; 6=2-Ethyl-2-hexenal; 8=2-Ethylhexanol, 10=2-ethylhexanal, 12=2,4-diethyl-2-octenal, 14=2,4-diethyloctanol, QH=quanternary ammonium or phosphonium hydroxide.

FIG. 4. A plot of C12/C8 products from the crossed-aldol reaction versus moles tributylmethylammonium chloride (PTC) normalized to moles of n-butyraldehyde (C4). C12=2,4-diethyl-2-octenal, C8=2-ethyl-2-hexenal (See Table 1).

FIG. 5. A plot of C12/C8 products from the crossed-aldol reaction versus moles of sodium hydroxide normalized to moles of n-butyraldehyde (C4). C12=2,3-diethyl-2-octenal, C8=2-ethyl-2-hexenal (See Table 1).

### DETAILED DESCRIPTION OF THE INVENTION

The present invention provides a process for the manufacture of an unsaturated aldehyde by the crossed-aldol condensation reaction of a first aldehyde containing 3-5 carbons and a second aldehyde containing 6-11 carbons followed by hydrogenation of the product aldehyde to an alcohol. Examples of first aldehyde can be, but are not limited to, propionaldehyde, n-butyraldehyde, isovaleraldehyde, or valeraldehyde.

Specifically, a process for the manufacture of 2,4-diethyloctanol by the crossed-aldol condensation reaction of n-butyraldehyde and 2-ethylhexanal followed by hydrogenation of the product aldehyde is described. The process of the current invention provides an improvement over prior art processes by making use of alkyl ammonium or phosphonium phase transfer catalysts (PTCs) that show limited solubility in aqueous alkali hydroxide solutions, but good solubility in water. In an alternative embodiment, 2-ethyl-2-hexenal can be used as a feed with n-butyraldehyde to produce 2,4-diethyl-2,4-octadienal which itself can ultimately be hydrogenated to 2,4-diethyloctanol.

The solubility characteristics of the PTCs preferred in the current process provide three advantages over previous processes. Firstly, the PTC/aldol catalysts are preferentially solvated in the organic aldehyde layer of the biphasic aldol reaction due to their poor solubility in aqueous solutions with high alkali metal hydroxide concentrations. This results in a much greater selectivity for the crossed-condensation reaction between n-butyraldehyde and 2-ethylhexanal, rather than the competing self-condensation reaction of n-butyraldehyde which predominates in the absence of PTCs. Secondly, the high water solubility of the PTCs used in the current invention allows for easy recovery of the PTCs from the organic product phase via aqueous washing. Finally, the solubility properties of the PTCs used in the current invention also allows for easy regeneration/recycle due to the fact that the PTCs can be induced to phase separate from the aqueous washings by the addition of an alkali metal hydroxide and recovered by decantation.

Preferably, the present invention is implemented in a process which modifies a 2-ethylhexanol plant to co-produce 2,4-diethyloctanol with a high selectivity for 2,4-diethyloctanol. A crossed-aldol reaction of n-butyraldehyde and 2-ethylhexanal is followed by the elimination of water to yield 2,4-diethyl-2-octenal and then hydrogenated to 2,4-diethyloctanol. During the crossed-aldol reaction, n-butyraldehyde can undergo a self-aldol condensation to yield 2-ethyl-2-hexenal and is ultimately hydrogenated to 2-ethylhexanol. In a refined embodiment of this invention, a portion of the 2-ethyl-2-hexenal and excess 2-ethylhexanal exiting the aldol section would be separated from the 2,4-diethyl-2-octenal stream by distillation and diverted back into the selective hydrogenation unit to be recycled into the 2-ethylhexanal feedstock stream.

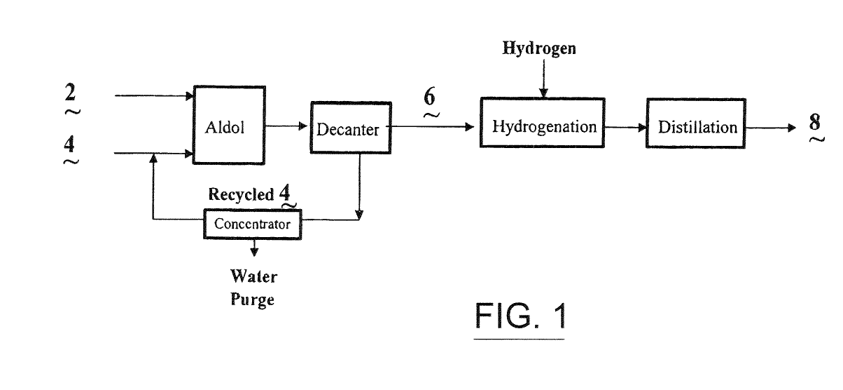

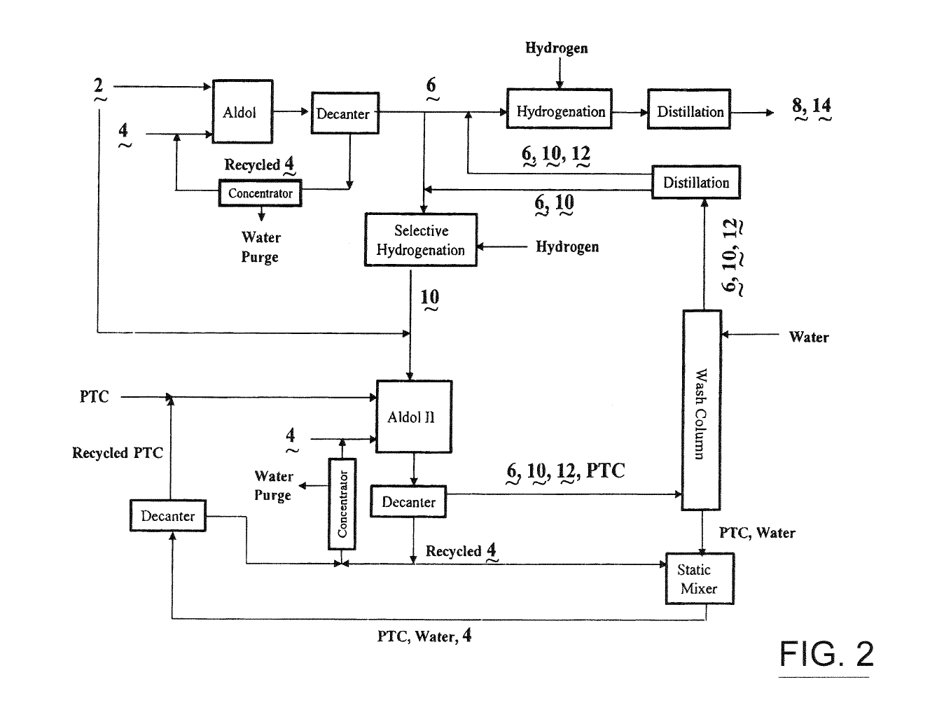

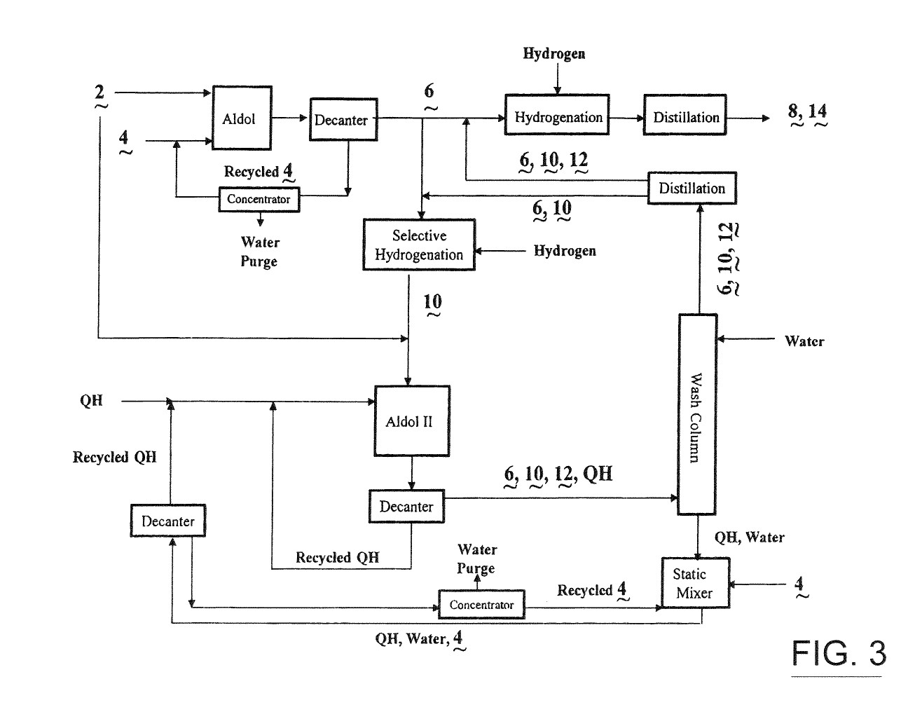

Referring to FIG. 1, a process flow diagram of the aldol and hydrogenation sections of a typical 2-ethylhexanol plant is shown, 2=n-butyraldehyde; 4=aqueous sodium hydroxide; 6=2-Ethyl-2-hexenal; 8=2-Ethylhexanol. Referring to FIGS. 2 and 3, two different modified process flows for the co-production of 2,4-diethyloctanol and 2-ethylhexanol are shown. Following the step for condensation of n-butyraldehyde to 2-ethyl-2-hexenal a step for selective hydrogenation produces 2-ethylhexanal (FIGS. 2 and 3). The process flow diagram in FIG. 2 shows a 2-ethylhexanol plant modified with a selective hydrogenation unit and phase transfer catalyst recycle unit, 10=2-ethylhexanal, 12=2,4-diethyl-2-octenal, 14=2,4-diethyloctanol, PTC=phase transfer catalyst. The 2-ethylhexanal is used as a co-feed into an aldol reactor with n-butyraldehyde to control the ratio of 2-ethylhexanol and 2,4-diethyloctanol (FIGS. 2 and 3) which is operated at a temperature from 30 to about 120° C. A water soluble quaternary ammonium or phosphonium salt as a phase transfer catalyst, hereinafter abbreviated to PTC, or a quaternary ammonium or phosphonium hydroxide is used as the aldol catalyst, hereinafter referred to as the “quaternary hydroxide”. In FIG. 3, a process flow diagram of a 2-ethylhexanol plant is shown modified with a selective hydrogenation unit and “quaternary hydroxide” recycle unit, QH=quanternary ammonium or phosphonium hydroxide. In a preferred embodiment those hydroxide forms that are soluble in the reacting aldehyde phase with a low solubility in a caustic solution are used. The amount of alkali metal hydroxide present in the aqueous phase of the aldol reaction can range from 0 to 50 wt. %. The PTC or quaternary hydroxide aldol catalyst is subsequently separated from the product aldehyde for re-use. The preferred method being the use of an aqueous stream to wash the PTC or quaternary hydroxide out of the product stream (FIGS. 2 and 3). Following extraction of the phase transfer catalyst or hydroxide catalyst from the product aldehyde, it can be recovered for reuse by addition of 10-50 wt. % alkali metal hydroxide (for example 2.5 to 12.4 molar sodium hydroxide) to the aqueous stream inducing phase separation of the quaternary ammonium or phosphonium hydroxide (FIGS. 2 and 3). The major difference in the processes depicted in FIGS. 2 and 3 are that the process in FIG. 3 uses quaternary hydroxides as aldol catalysts and only uses the alkali metal hydroxide solutions to recycle the quaternary hydroxides from the water washings. The process in FIG. 2 also uses the alkali metal hydroxide solution in the aldol to generate the quaternary hydroxide in situ from a quaternary halide and/or reduce the solubility of the quaternary hydroxide in the aqueous phase in the aldol reaction.

In the preferred embodiment of this invention, selective hydrogenation is used to convert 2-ethyl-2-hexenal aldol product to 2-ethylhexanal in a 2-ethylhexanol plant modified to co-produce 2,4-diethyloctanol. This selective hydrogenation can be performed by a Group VIII metal catalyst, preferably palladium. 2-ethylbexanal is then fed to an aldol reactor and reacted with n-butyraldehyde. 2-ethyl-2-hexenal could be used in place of 2-ethylhexanal in a crossed aldol reaction with n-butyraldehyde to yield 2,4-diethyl-2,4-octadienal which itself can ultimately be hydrogenated to 2,4-diethyloctanol. The route through 2-ethylhexanal is preferred as it minimizes heavies formation.

The commercialized aldol reaction of butyraldehyde to 2-ethyl-2-hexenal is carried out in a biphasic reaction. The two phases of the reaction are the reactant/product aldehyde layer and an aqueous sodium hydroxide layer which catalyzes the reaction and takes up the water of reaction. Unique improvement to the aldolization unit is the use of selective aldol catalysts which are water soluble quaternary ammonium hydroxides or quaternary phosphonium hydroxides which are also readily soluble in aldehyde mixtures. The hydroxides can be generated from their respective anion salts with the most common anions being chloride or bromide. Other suitable anions are iodine, sulfates, bisulfates, phosphates, etc. The hydroxides are then generated by anion exchange with an alkali metal hydroxide in aqueous solution, preferably sodium hydroxide. The water solubility of the PTC is essential to separate the PTC from the product stream via aqueous extraction for recycle. Separation by distillation is not a viable option for most PTC's as they are unstable at high temperatures.

Water soluble catalysts which have yielded enhanced selectivity's equal to or better than the slightly water soluble tricaprylmethylammonium chloride PTC are: hexadecyltrimethylammonium chloride, tetrabutylammonium chloride, tetrabuytlammonium bromide, tributymethylammonium chloride, tetrabuylammonium hydroxide, tetrabutylphosphonium chloride, and benzyltriethylammonium chloride. Tetramethylammonium chloride gave an unsatisfactory performance.

Benzyltriethylammonium chloride showed no yield improvement versus tricaprylmethylammonium chloride however, it forms a third-phase in the reaction. Formation of such a third-phase has been shown to be advantageous for catalyst recycle (Starks et al. Phase-Transfer Catalysis, 1994). Despite the lower yields observed for this PTC, its third-phase behavior could allow for the development of a higher efficiency process due to ease of separation.

TABLE 1
Examples of water soluble PTC's for the selective production of 2,4-diethyl-2-octenal
(C12) vs 2-ethyl-2-hexenal (C8) from n-butyraldehyde (C4) and 2-ethylhexanal.

| | | PTC/C4 | | | | C12/C8 |
|---|---|---|---|---|---|---|
| | | molar | NaOH/C4 | C12 % | C8 % | molar |
| Example | PTC | ratio | molar ratio | Yield | Yield | ratio |
| Comparative | None | 0 | 1.40 | 4.7 | 28.7 | 0.2 |
| example 1 | | | | | | |
| Comparative | Tricaprylmethyl- | 0.09 | 1.44 | 42.3 | 24.6 | 1.7 |
| example 2 | ammonium chloride | | | | | |
| Comparative | Tetramethylammonium | 0.21 | 1.34 | 17.0 | 33.9 | 0.5 |
| example 3 | chloride | | | | | |
| Example 1 | Hexadecyltrimethyl- | 0.08 | 1.36 | 47.4 | 18.1 | 2.6 |
| | ammonium chloride | | | | | |
| Example 2 | Tetrabutylammonium | 0.09 | 1.36 | 57.5 | 13.1 | 4.4 |
| | chloride | | | | | |
| Example 3 | Tetrabutylammonium | 0.10 | 1.29 | 58.6 | 15.4 | 3.8 |
| | bromide | | | | | |
| Example 4 | Tributylmethyl- | 0.07 | 1.26 | 61.1 | 9.6 | 6.4 |
| | ammonium chloride | | | | | |
| Example 5 | Tetrabutylammonium | 0.09 | 1.34 | 61.7 | 11.2 | 5.5 |
| | hydroxide | | | | | |
| Example 6 | Tributylmethyl- | 0.04 | 1.23 | 51.8 | 21.2 | 2.4 |
| | ammonium chloride | | | | | |
| Example 7 | Tributylmethyl- | 0.02 | 1.44 | 38.8 | 32.5 | 1.2 |
| | ammonium chloride | | | | | |
| Example 8 | Tributylmethyl- | 0.09 | 0.69 | 41.3 | 30.1 | 1.4 |
| | ammonium chloride | | | | | |
| Example 9 | Tributylmethyl- | 0.09 | 0.35 | 20.0 | 57.4 | 0.3 |
| | ammonium chloride | | | | | |
| Example 10 | Tributylmethyl- | 0.09 | 2.13 | 52.6 | 7.6 | 6.9 |
| | ammonium chloride | | | | | |
| Example 11 | Tetrabutylammonium | 0.09 | 0.00 | 52.7 | 22.2 | 2.4 |
| | hydroxide | | | | | |
| Example 12 | Tetrabutylphosphonium | 0.10 | 0.00 | 40.0 | 40.6 | 1.0 |
| | hydroxide | | | | | |
| Example 13 | Benzyltriethyl- | 0.10 | 1.31 | 41.4 | 27.6 | 1.5 |
| | ammonium chloride | | | | | |

The preferred quaternary cations for the aldol reaction are tetrabutylammonium and tributylmethylammonium which gave molar yields of over 60% 2,4-diethyl-2-octenal based on n-butyraldehyde consumed in the reaction with a molar excess of 2-ethylhexanal at 60° C. The remaining n-butyraldehyde gave 2-ethyl-2-hexenal and undesirable byproducts. The halide salts of these cations can be used as PTC's with an aqueous alkali metal hydroxide layer. The alkali metal not only generates the hydroxide form but also decreases the solubility of the hydroxide in the aqueous phase, thereby increasing its concentration in the aldehyde phase. Both the tetrabutylammonium hydroxide and tributylmethylammonium hydroxide can also be used directly as aldol catalysts for the desired reaction. The addition of some alkali metal hydroxide to the aqueous phase (10-30%) is also desirable to decrease the solubility of the quaternary hydroxides in the aqueous phase.

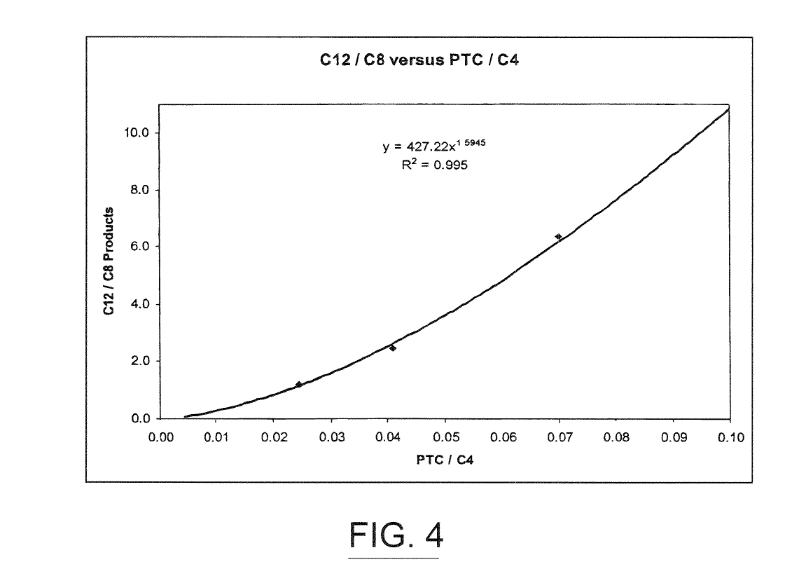

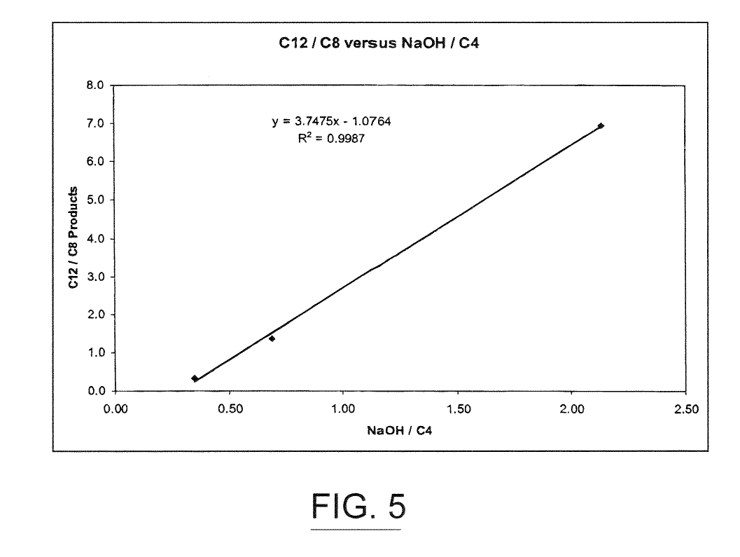

It is proposed that an increase in selectivity for the reaction of n-butyraldehyde and 2-ethylhexanal is affected by this increase in hydroxide concentration and the steric bulk of the accompanying quaternary cation. The quaternary ammonium or phosphonium cations of these catalysts increase the effective concentration of the hydroxide anion in the mixed aldehyde reactants. In the absence of quaternary cations, a significant portion of the reaction may occur in the aqueous phase in which the n-butyraldehyde is more soluble (than 2-ethylhexanal) and therefore more likely to undergo a self-condensation reaction. It has been found that the PTC concentration {the molar ratio of PTC/n-butyraldehyde (C4)} drastically effects the ratio of 2,4-diethyl-2-octenal (C12) to 2-ethyl-2-hexenal (C8) ratio in the product aldehyde stream (See Table 1), In FIG. 4, a plot of C12/C8 products from the crossed-aldol reaction versus moles tributylmethylammonium chloride (PTC) normalized to moles of n-butyraldehyde (C4) is shown (C12=2,3-diethyl-2-octenal, C8=2-ethyl-2-hexenal). This makes the PTC concentration a very important variable in the invention which is contrary to the prior art (GB1547856) which states “The exact amount of PTC is not critical because a finite amount of said catalyst produces a finite increase in the amoun of product aldehyde formed.” The preferred embodiment would make use of a PTC/n-butyraldehyde ratio of 0.01 to 0.2. A similar effect is observed for the concentration of sodium hydroxide in the reaction. As depicted in FIG. 5, a plot of C12/C8 products from the crossed-aldol reaction is shown versus moles of sodium hydroxide normalized to moles of n-butyraldehyde (C4). C12=2,3-diethyl-2-octenal, C8=2-ethyl-2-hexenal (See Table 1).

The water solubility of the tetrabutylammonium hydroxide and tributylmethylammonium salts makes them ideal for recovery by aqueous extraction. Tributylmethylammonium chloride has been reported to provide excellent recoverability from organic phases and is recoverable from dilute aqueous streams by increasing the hydroxide concentration of the aqueous stream to “salt out” the ammonium hydroxide (Halpern and Grinstein, Spec. Publ. -R. Soc. Chem., 1999, 30-39).

Heterogeneous hydrogenation catalysts which are used for the commercial hydrogenation of 2-ethyl-2-hexenal to 2-ethylhexanol work well to prepare 2,4-diethyloctanol from 2,4-diethyl-2-octenal. Excess 2-ethylhexanal and 2-ethyl-2-hexenal co-produced in the aldol reaction would then be hydrogenated to the 2-ethylhexanol co-product. The co-produced aldehydes can be separated prior to hydrogenation by distillation, or separated as alcohols by distillation after the hydrogenation. After purification by distillation, 2,4-diethyloctanol can be esterified with phthalic anhydride to produce bis(2,4-diethyloctyl)phthalate. PVC sheet compounded with this plasticizer demonstrates properties superior to the similar plasticizer prepared from isodecyl alcohol and ortho-phthalic anhydride. Other desirable plasticizers can be produced with other acids and anhydrides, such as terephthalic, isophthalic, maleic, adipic, and trimellitic acids or anhydrides. Useful co-esters can be prepared from such multifunctional acids or anhydrides and blends of other common oxo alcohols and 2,4-diethyloctanol. Such co-esters produced from esterification and/or transesterification can be used to “tune” the desired properties of the resulting plasticizers. Esters for non-plasticizer uses, such as diethyloctyl methacrylate, can also be prepared.

The preferred embodiment of this invention makes use of a water soluble quaternary ammonium salt as a PTC or a water soluble quaternary ammonium hydroxide as direct aldol catalyst which is selective for the crossed-aldol reaction of butanal and 2-ethylhexanal to co-produce 2,4-diethyl-2-octenal with 2-ethyl-2-hexenal co-product. The resulting co-products from the hydrogenation and separation of the resulting mixed aldehyde stream are the highly desirable plasticizer alcohol 2,4-diethyloctanol and 2-ethylhexanol.

The following examples are illustrative of the present invention and should not be regarded as restrictive: The aldol reactions were performed under an inert atmosphere of nitrogen. The aldehyde mixtures were added drop wise over the course of an hour to a mixture of aqueous sodium hydroxide and/or quaternary ammonium/phosphonium compounds. The reaction was stirred at 60° C. for approximately an hour. The co-product organic layer was then separated from the aqueous layer and water washed. Co-product yields were determined by gas chromatography and are expressed as the molar yield of each co-product based on the initial weight of n-butyraldehyde.

### Comparative Example 1

A 3-neck 1-liter flask was charged with 58.5 grams of sodium hydroxide and 133.3 grams of deionized water. To this mixture, an aldehyde solution of 261.8 grams of 2-ethylhexanal and 75.0 grams of n-butyraldehyde was added drop wise. The organic layer was water washed and analyzed by gas chromatography. Yields were calculated to be 4.7% 2,4-diethyl-2-octenal and 28.7% 2-ethyl-2-hexenal.

### Comparative Example 2

A 3-neck 1-liter flask was charged with 36.2 grams of tricaprylmethyl-ammonium chloride (Aliquat® 336), 58.1 grams of sodium hydroxide and 138.6 grams of deionized water. To this mixture, an aldehyde solution of 265.0 grams of 2-ethylhexanal and 72.7 grams of n-butyraldehyde was added drop wise. The organic layer was water washed and analyzed by gas chromatography. Yields were calculated to be 42.3% 2,4-diethyl-2-octenal and 24.6% 2-ethyl-2-hexenal.

### Comparative Example 3

A 3-neck 1-liter flask was charged with 24.6 grams of tetramethylammonium chloride, 56.2 grams of sodium hydroxide and 132.7 grams of deionized water. To this mixture, an aldehyde solution of 259.3 grams of 2-ethylhexanal and 75.3 grams of n-butyraldehyde was added drop wise. The organic layer was water washed and analyzed by gas chromatography. Yields from n-butyraldehyde were calculated to be 17.0° A) 2,4-diethyl-2-octenal and 33.9% 2-ethyl-2-hexenal.

### Example 1

A 3-neck 1-liter flask was charged with 25.7 grams of hexadecyltrimethylammonium chloride, 56.3 grams of sodium hydroxide and 132.9 grams of deionized water. To this mixture, an aldehyde solution of 265.4 grams of 2-ethylhexanal and 74.4 grams of n-butyraldehyde was added drop wise. The organic layer was water washed and analyzed by gas chromatography. Yields from n-butyraldehyde were calculated to be 47.4% 2,4-diethyl-2-octenal and 18.1% 2-ethyl-2-hexenal.

### Example 2

A 3-neck 1-liter flask was charged with 25.0 grams of tetrabutylammonium chloride, 56.7 grams of sodium hydroxide and 132.7 grams of deionized water. To this mixture, an aldehyde solution of 257.2 grams of 2-ethylhexanal and 75.1 grams of n-butyraldehyde was added drop wise. The organic layer was water washed and analyzed by gas chromatography. Yields from n-butyraldehyde were calculated to be 57.5% 2,4-diethyl-2-octenal and 13.1% 2-ethyl-2-hexenal.

### Example 3

A 3-neck 1-liter flask was charged with 29.9 grams of tetrabutylammonium bromide, 56.4 grams of sodium hydroxide and 132.7 grams of deionized water. To this mixture, an aldehyde solution of 260.0 grams of 2-ethylhexanal and 78.6 grams of n-butyraldehyde was added drop wise. The organic layer was water washed and analyzed by gas chromatography. Yields from n-butyraldehyde were calculated to be 58.6% 2,4-diethyl-2-octenal and 15.4% 2-ethyl-2-hexenal.

### Example 4

A 3-neck 1-liter flask was charged with 28.8 grains of tributylmethylammonium chloride (75 wt. % in water), 56.5 grams of sodium hydroxide and 132.5 grams of deionized water. To this mixture, an aldehyde solution of 255.9 grams of 2-ethylhexanal and 80.7 grams of n-butyraldehyde was added drop wise. The organic layer was water washed and analyzed by aas chromatography. Yields from n-butyraldehyde were calculated to be 61.1% 2,4-diethyl-2-octenal and 9.6% 2-ethyl-2-hexenal.

### Example 5

A 3-neck 1-liter flask was charged with 59.2 grams of tetrabutylammonium hydroxide (40 wt. % in water), 57.0 grams of sodium hydroxide and 132.5 grams of deionized water. To this mixture, an aldehyde solution of 259.9 grams of 2-ethylhexanal and 76.8 grams of n-butyraldehyde was added drop wise. The organic layer was water washed and analyzed by gas chromatography. Yields from n-butyraldehyde were calculated to be 61.7% 2,4-diethyl-2-octenal and 11.2% 2-ethyl-2-hexenal.

### Example 6

A 3-neck 1-liter flask was charged with 14.9 grams of tributylmethylammonium chloride, 56.7 grams of sodium hydroxide and 132.5 grams of deionized water. To this mixture, an aldehyde solution of 257.8 gams of 2-ethylhexanal and 83.3 grams of n-butyraldehyde was added drop wise. The organic layer was water washed and analyzed by gas chromatography. Yields from n-butyraldehyde were calculated to be 51.8% 2,4-diethyl-2-octenal and 21.2% 2-ethyl-2-hexenal.

### Example 7

A 3-neck 1-liter flask was charged with 7.7 grams of tributylmethylammonium chloride, 57.5 grams of sodium hydroxide and 131.9 grams of deionized water. To this mixture, an aldehyde solution of 241.7 grams of 2-ethylhexanal and 71.9 grams of n-butyraldehyde was added drop wise. The organic layer was water washed and analyzed by gas chromatography. Yields from n-butyraldehyde were calculated to be 38.8% 2,4-diethyl-2-octenal and 32.5% 2-ethyl-2-hexenal.

### Example 8

A 3-neck 1-liter flask was charged with 28.5 grams of tributylmethylammonium chloride, 28.7 grams of sodium hydroxide and 141.5 grams of deionized water. To this mixture, an aldehyde solution of 257.4 grams of 2-ethylhexanal and 75.2 grams of n-butyraldehyde was added drop wise. The organic layer was water washed and analyzed by gas chromatography. Yields from n-butyraldehyde were calculated to be 41.3% 2,4-diethyl-2-octenal and 30.1% 2-ethyl-2-hexenal.

### Example 9

A 3-neck 1-liter flask was charged with 28.7 grams of tributylmethylammonium chloride, 14.3 grams of sodium hydroxide and 133.0 grams of deionized water. To this mixture, an aldehyde solution of 257.6 grams of 2-ethylhexanal and 73.5 grams of n-butyraldehyde was added drop wise. The organic layer was water washed and analyzed by gas chromatography. Yields from n-butyraldehyde were calculated to be 20.0% 2,4-diethyl-2-octenal and 57.4% 2-ethyl-2-hexenal.

### Example 10

A 3-neck 1-liter flask was charged with 28.6 grams of tributylmethylammonium chloride, 85.7 grams of sodium hydroxide and 133.0 grams of deionized water. To this mixture, an aldehyde solution of 256.0 grams of 2-ethylhexanal and 72.3 grams of n-butyraldehyde was added drop wise. The organic layer was water washed and analyzed by gas chromatography. Yields from n-butyraldehyde were calculated to be 52.6% 2,4-diethyl-2-octenal and 7.6% 2-ethyl-2-hexenal.

### Example 11

A 3-neck 1-liter flask was charged with 59.8 grams of tetrabutylammonium hydroxide (40 wt. % in water). To this solution, an aldehyde solution of 276.0 grams of 2-ethylhexanal and 76.0 grams of n-butyraldehyde was added drop wise. The organic layer was water washed and analyzed by gas chromatography. Yields from n-butyraldehyde were calculated to be 52.7% 2,4-diethyl-2-octenal and 22.2% 2-ethyl-2-hexenal.

### Example 12

A 3-neck 1-liter flask was charged with 70.08 grams of tetrabutylphosphonium hydroxide (40 wt. % in water). To this solution, an aldehyde solution of 255.9 grams of 2-ethylhexanal and 72.2 grams of n-butyraldehyde was added drop wise. The organic layer was water washed and analyzed by gas chromatography. Yields from n-butyraldehyde were calculated to be 40.0% 2,4-diethyl-2-octenal and 40.6% 2-ethyl-2-hexenal.

### Example 13

A 3-neck 1-liter flask was charged with 25.0 grams of benzyltriethylammonium chloride, 56.4 grams of sodium hydroxide and 132.5 grams of deionized water. To this mixture, an aldehyde solution of 258.2 grams of 2-ethylhexanal and 77.6 grams of n-butyraldehyde was added drop wise. The organic layer was water washed and analyzed by gas chromatography. Yields from n-butyraldehyde were calculated to be 41.4% 2,4-diethyl-2-octenal and 27.6% 2-ethyl-2-hexenal.

### Examples 14-19

A continuous aldol loop was constructed from ⅜ inch stainless steel tubing and an 11.5 inch segment, which contained 34 fixed right- and left-band elements to insure turbulent flow. The contents of the loop were circulated using a centrifugal pump with a 316 stainless steel pump body with Teflon® bushing and o-rings. The aldol loop was heat traced for temperature control and had a total inner volume of 75 ml. Up to four metering pumps were used to introduce reagents into the aldol loop, which was also equipped with a backpressure valve set to 40 psig, which allowed for the displacement of aldol product out of the loop. Various conditions for the aldol reaction (See Table 2) were examined using the continuous reactor. The product was sampled, water-washed, and analyzed by gas chromatography (See Table 3). DEO Yields are calculated based on nBal conversion assuming the mass of the organic product is the sum of the aldehydes minus the weight of one mole of water per mole n-butyraldehyde. The product was allowed to separate from the aqueous layer and was decanted into a counter-current aqueous wash column. The column contained approximately two liters of deionized, deaerated water and was fed with a ratio of 1 volume of deionized, deaerated water for every 3 volumes of organic product. The resultant washed product was collected and stored under an argon atmosphere. The product contained between 0.04 and 1.6 wt. % methyltributyl-ammonium cation (chloride salt equivalents) determined by titration with sodium tetraphenylborate.

TABLE 2
Experimental conditions for examples 14-19

| | | | | 75% | | 2EHal/nBal | PTC/nBal | NaOH/nBal |
|---|---|---|---|---|---|---|---|---|
| | Temp | nBal | 2EHal | MTBACl | 30% NaOH | mole | mole | mole |
| Example | ° C. | ml/min | ml/min | ml/min | ml/min | ratio | ratio | ratio |
| 14 | 93 | 1.67 | 5.45 | 0.52 | 0.19 | 1.86 | 0.08 | 0.1 |
| 15 | 93 | 1.25 | 6.82 | 0.57 | 0.15 | 3.1 | 0.12 | 0.1 |
| 16 | 113 | 1.00 | 4.60 | 0.47 | 1.50 | 2.62 | 0.13 | 1.30 |
| 17 | 82 | 2.07 | 3.64 | 0.64 | 2.00 | 1.00 | 0.08 | 0.83 |
| 18 | 98 | 2.08 | 3.64 | 5.72 | 0.63 | 1.00 | 0.08 | 0.83 |
| 19 | 99 | 5.3 | 2.00 | 0.48 | 0.2 | 2.00 | 0.02 | 0.03 |

nBal = n-butyraldehyde,
2EHal = 2-ethylhexanal,
NaOH = sodium hydroxide,
PTC = MTBACl = methyltributylammonium chloride.

TABLE 3
Analysis of the product produced in continuous aldol Examples 14-19.

| | nBal | 2EHal | 2E2H | DEO | Heavies | DEO Yield |
|---|---|---|---|---|---|---|
| Example | Area % | Area % | Area % | Area % | Area % | % Calc. |
| 14 | 0.09 | 62.04 | 8.55 | 22.55 | 6.17 | 36.1 |
| 15 | 0.14 | 70.23 | 7.41 | 19.36 | 2.57 | 48.0 |
| 16 | 0.06 | 69.55 | 3.37 | 19.8 | 5.71 | 42.3 |
| 17 | 0.09 | 39.10 | 6.80 | 33.44 | 19.01 | 33.4 |
| 18 | 0.07 | 41.87 | 8.39 | 30.16 | 15.84 | 30.2 |
| 19 | 0.34 | 62.74 | 13.15 | 17.02 | 3.44 | 29.0 |

DEO = 2,4-diethyl-2-octenal;
Heavies are defined as the sum of any peaks eluting with a retention time greater than DEO by gas chromatography.
DEO Yields are calculated based on nBal conversion.

### Examples 20-22

To simulate recovery of methyltributylammonium chloride (MTBACl) from product washings, enough sodium hydroxide (NaOH) was added to an aqueous solution containing 17.7 wt. % MTBACl to make a 9.9% wt. NaOH solution (Wt. % based only on water and NaOH). No second phase was formed. Upon further addition of NaOH to make a 12% NaOH solution, the second phase was still absent. A second phase formed above the NaOH solution after enough NaOH was added to make a 25 wt. % NaOH solution. The titration of this MTBACl phase with a sodium tetraphenylborate solution indicated a 97% recovery of methyltributylammonium cation.

### Example 23

To simulate recovery of methyltributylammonium chloride (MTBACl) from product washings, enough sodium hydroxide (NaOH) was added to an aqueous solution containing 17.7 wt. % MTBACl to make a 40% wt. NaOH solution and form a second phase. Titration of the MTBACl phase showed only a 78% recovery implying there was some decomposition of the MTBACl.

### Example 24

To simulate recovery of methyltributylammonium chloride (MTBACl) from product washings, enough sodium hydroxide (NaOH) was added to an aqueous solution containing 17.7 wt. % MTBACl to make a 50% wt. NaOH solution and form a second phase. Titration of the MTBACl phase showed only a 61% recovery implying there was some decomposition of the MTBACl.

### Example 25

To simulate recovery of methyltributylammonium chloride (MTBACl) from product washings, enough sodium hydroxide (NaOH) was added to an aqueous solution containing 21.5 wt. % MTBACl to make a 15.2% wt. NaOH solution and form a second phase. Titration of the MTBACl phase showed a 76% recovery of methyltributylammonium cation.

### Example 26

To simulate recovery of methyltributylammonium chloride (MTBACl) from product washings, enough sodium hydroxide (NaOH) was added to an aqueous solution containing 17.7 wt. % MTBACl to make a 20.0% wt. NaOH solution and form a second phase. Titration of the MTBACl phase showed a 92% recovery of methyltributylammonium cation.

### Example 27

To simulate recovery of methyltributylammonium chloride (MTBACl) from product washings, enough sodium hydroxide (NaOH) was added to an aqueous solution containing 17.7 wt. % MTBACl to make a 30.0% wt. NaOH solution and form a second phase. Titration of the MTBACl phase showed a 93% recovery of methyltributylammonium cation.

TABLE 4
Examples 20-27. Recovery of methyltributylammonium chloride from
aqueous solutions using sodium hydroxide.

| | Moles | Wt % | Moles PTC | % |
|---|---|---|---|---|
| Example | MTBACl | NaOH* | Recovered | Recovery |
| 20 | 0.0197 | 9.9% | 0.0000 | 0 |
| 21 | 0.0197 | 12.0% | 0.0000 | 0 |
| 22 | 0.0197 | 25.0% | 0.0190 | 97% |
| 23 | 0.0197 | 40.0% | 0.0154 | 78% |
| 24 | 0.0197 | 50.0% | 0.0120 | 61% |
| 25 | 0.0328 | 15.2% | 0.0249 | 76% |
| 26 | 0.0328 | 20.0% | 0.0302 | 92% |
| 27 | 0.0328 | 30.0% | 0.0307 | 93% |

*Based on sodium hydroxide and water.

### Example 28

A composite sample of product from the continuous aldol product was hydrogenated in an upward flow gas-phase reactor, which had an internal diameter of 26 mm with a height of 86 cm. The reactor was filled with a commercial copper-zinc hydrogenation catalyst in the form of 9.5 mm pellets. Typical feed rates were 2 ml/min of aldol product and 8.0 standard liters of hydrogen per minute at a pressure of 80 psig and temperature between 150-200° C. A pre-heated vaporizer was employed at a temperature of 182° C., to vaporize 2,4-diethyl-2-octenal while knocking out heavies into the vaporizer bottoms. Analysis of the product and feed are shown in Table 5.

TABLE 5
Hydrogenation feed and product analysis.

| | | | | 2,4- | 2,4- | | |
|---|---|---|---|---|---|---|---|
| | 2- | 2-ethyl-2- | 2-ethyl- | diethyl-2- | diethyl- | 2-ethylhexyl 2- | |
| | ethylhexanal | hexenal | hexanol | octenal | octanol | ethylhexanoate | *Heavies |
| | % area | % area | % area | % area | % area | % area | % area |
| Feed | 60.2 | 8.1 | | 18.0 | | | 11.1 |
| Product | 0.15 | | 77.2 | | 18.7 | 0.96 | 1.66 |

*Heavies are defined as all compounds eluting with a retention time longer than 2,4-diethyloctanol other than 2-ethylhexyl 2-ethylhexanoate.

1.-24. (canceled)

25. A continuous process for the optimized coproduction of 2,4-diethyloctanol in a 2-ethylhexanol manufacturing process comprising the steps of:
(a) forming an unsaturated aldehyde reaction product mixture comprised of 2-ethyl-2-hexenal and 2,4-diethyl-2-octenal by an aqueous base-catalyzed crossed-aldol condensation reaction between n-butyraldehyde and 2-ethylhexanal in the presence of (i) a quaternary ammonium or phosphonium salt as a water soluble phase-transfer catalyst (PTC) which is present in a molar ratio of the PTC to the n-butyraldehyde of between 0.01:1 to 0.2:1, and (ii) an alkali metal hydroxide in a molar ratio of the alkali metal hydroxide to the n-butyraldehyde of between 0.03:1 to 1.30:1,
(b) separating the PTC from the aldehyde reaction product mixture by washing a stream of the unsaturated aldehyde reaction product mixture with a water stream to obtain an aqueous PTC-containing stream and an organic unsaturated aldehyde product-containing stream;
(c) subjecting the unsaturated aldehyde reaction product-containing stream obtained in step (b) to hydrogenation to form a product stream comprised of 2-ethylhexanol and 2,4-diethyloctanol;
(d) recovering the PTC from the PTC-containing stream obtained in step (b) by adding an alkali metal hydroxide solution to the aqueous PTC-containing stream to thereby form a first aqueous phase containing the sodium hydroxide and a second phase containing predominantly the PTC, whereby the PTC is recovered in the second phase; and
(e) recycling the PTC recovered in step (d) to the crossed-aldol condensation reaction of step (a).

26. The process of claim 25, wherein the aldol reaction is performed at a temperature from about 30 to 120° C.

27. The process of claim 25, wherein the aldol reaction is performed at a temperature from about 30 to about 100° C.

28. The process of claim 25, wherein the sodium hydroxide solution comprises a 10-50 weight percent solution of sodium hydroxide.
